(19) United States
(12) Patent Application Publication
(10) Pub. No.: US 2015/0099913 A1
(43) Pub. Date: Apr. 9, 2015

(54) METHANOL CONVERSION PROCESS

(71) Applicants: Luc Roger Marc MARTENS, Meise (BE); David M. MARCUS, New Albany, OH (US); Teng XU, Houston, TX (US)

(72) Inventors: Luc Roger Marc MARTENS, Meise (BE); David M. MARCUS, New Albany, OH (US); Teng XU, Houston, TX (US)

(73) Assignee: ExxonMobil Research and Engineering Company, Annandale, NJ (US)

(21) Appl. No.: 14/046,548

(22) Filed: Oct. 4, 2013

(51) Int. Cl. C07C 1/22

(52) U.S. Cl. CPC C07C 1/22
USPC 585/408; 585/640

(57) ABSTRACT

A methanol conversion process comprises contacting a feedstream comprising methanol, optionally with dimethyl ether or other oxygenates with a catalyst comprising a physical mixture of a molecular sieve, preferably an intermediate or small pore size zeolite such as an MFI zeolite, with a basic metal oxide to provide extended catalyst cycle life by reducing the incidence of coke formation. The process may be applied to the methanol-to-gasoline (MTG), methanol to distillate (MOD), methanol-to-olefins (MTO), methanol-to-chemicals (MTC) and combination processes such as the MTO/OCP Process.

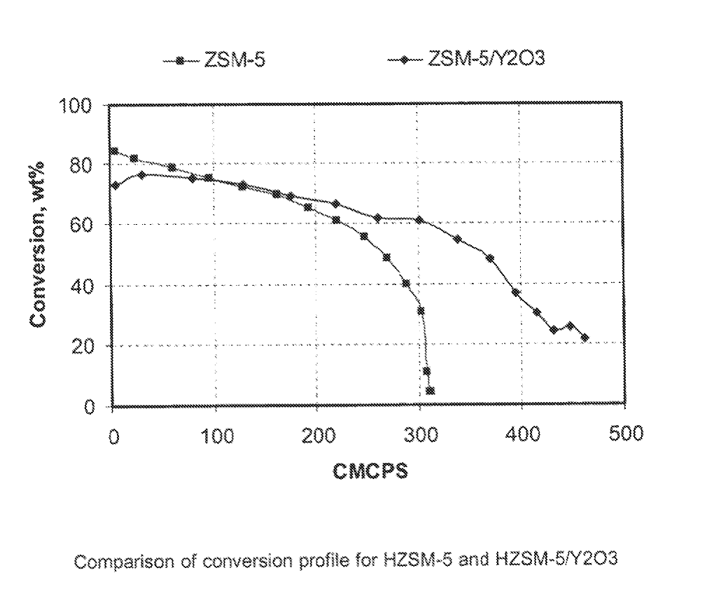

### FIELD OF THE INVENTION

This invention relates to a process for the conversion of methanol or mixtures of methanol with dimethyl ether to hydrocarbon chemicals such as light olefins, gasoline, distillates and aromatics useful as fuel blend stocks or as petrochemical feeds.

### BACKGROUND OF THE INVENTION

Olefins are traditionally produced from petroleum feedstocks by catalytic or steam cracking processes. These cracking processes, especially steam cracking, produce light olefin(s), such as ethylene and/or propylene, from a variety of hydrocarbon feedstocks. Ethylene and propylene are important commodity petrochemicals useful in a variety of processes for making plastics and other chemical compounds. With the increasing cost of petroleum crudes, oxygenates, especially alcohols, have entered into use for conversion into various hydrocarbon chemicals including light olefins such as ethylene and propylene, gasoline and distillate boiling range hydrocarbons. There are numerous technologies available for producing oxygenates including fermentation or reaction of synthesis gas derived from natural gas, petroleum liquids or carbonaceous materials including coal, recycled plastics, municipal waste or any other organic material. Generally, the production of synthesis gas involves a combustion reaction of natural gas, mostly methane, and an oxygen source into hydrogen, carbon monoxide and/or carbon dioxide. Other known syngas production processes include conventional steam reforming, autothermal reforming, or a combination of these processes.

Methanol, the preferred alcohol for light olefin production, is typically synthesized from the catalytic reaction of hydrogen, carbon monoxide and/or carbon dioxide in a methanol reactor in the presence of a heterogeneous catalyst. For example, in one synthesis process methanol is produced using a copper/zinc oxide catalyst in a water-cooled tubular methanol reactor. The preferred process for converting a feedstock containing methanol into one or more olefin(s), primarily ethylene and/or propylene, typically contacting the feedstock with a molecular sieve catalyst composition.

Various commercial processes have evolved using these and related technologies. The ExxonMobil MTG (Methanol-to-Gasoline) process, (MTG) was developed in the 1970's and first commercialized the technology in a fixed bed process which was later developed into fluidized bed applications with extensions into the production of olefins (the MTO Process) and chemicals (MTC Process). Other companies including UOP and Total have also been active in this area: a useful summary of methanol conversion technologies is given in Methanol to Olefins (MTO): Development of a Commercial Catalytic Process, Simon R. Bare, Advanced Characterization, UOP LLC, Modern Methods in Heterogeneous Catalysis Research, FHI Lecture 30 Nov. 2007 (©2007 UOP LLC, All Rights Reserved).

The MTO/OCP Process (Methanol-to Olefins/Olefin Cracking Process) developed jointly by UOP and Total combines the MTO process and the olefin cracking process to convert heavier olefins in the C4 to C8 range olefins by rearrangement in the presence of methanol via oligomerization-cracking and alkylation to form a product enriched in lighter olefins (additional ethylene and propylene) for polymerization into polyethylene and polypropylene. The conversion of methanol to aromatics over a modified zeolite catalyst is also known, as in U.S. Pat. No. 8,450,548 (Karim).

These variants of the basic methanol conversion process technology rely upon the conversion of methanol or its primary dehydration product, dimethyl ether, into hydrocarbons over a molecular sieve. There are many different types of molecular sieve well known to convert oxygenate feedstocks, into one or more olefin(s and other hydrocarbons. For example, U.S. Pat. No. 5,367,100 describes the use of the zeolite, ZSM-5, to convert methanol into olefin(s); U.S. Pat. No. 4,062,905 discusses the conversion of methanol and other oxygenates to ethylene and propylene using crystalline aluminosilicate zeolites, for example Zeolite T, ZK5, erionite and chabazite; U.S. Pat. No. 4,079,095 describes the use of ZSM-34 to convert methanol to hydrocarbon products such as ethylene and propylene; and U.S. Pat. No. 4,310,440 describes producing light olefin(s) from an alcohol using a crystalline aluminophosphate, often designated AIPO4. Among the many other patents describing methanol conversion over zeolite molecular sieve catalysts are, for example, U.S. Pat. No. 4,049,573 (boron or magnesium modified intermediate pore zeolites), U.S. Pat. No. 4,547,602 (two stage process using intermediate pore size zeolites), U.S. Pat. No. 5,367,100 (ZSM-5 catalyst modified with phosphorus, rare earth), U.S. Pat. No. 6,372,949 (unidimensional intermediate pore size zeolite catalyst), U.S. Pat. No. 6,740,790 (SAPO catalyst using a catalyst feedstock expore index of at least 1.0), U.S. Pat. No. 6,743,747 (SAPO catalyst with preference for SAPO-340, EP 083160 (small pore size zeolite modified with magnesium oxide manganese oxide or magnesium oxide and platinum oxide), US 2006/0025644 (catalyst comprises a molecular sieve and at least one metal oxide having an uptake of carbon dioxide at 100° C. of at least 0.03 mg/m² of the metal oxide); US 2007/0244000 (two-component catalyst of a metal oxide and a molecular sieve for methanol conversion followed by a molecular sieve for olefin formation), WO 98/29370 (small pore non-zeolitic molecular sieve containing a lanthanide, actinide, scandium, yttrium, a Group 4 metal or a Group 5 metal).

Typically, molecular sieves are formed into molecular sieve catalyst compositions to improve their durability in commercial conversion processes. These molecular sieve catalyst compositions are formed by combining the molecular sieve with a matrix material and/or a binder, which typically are clays or metal oxides. However, these binders and matrix materials typically only serve to provide desired physical characteristics to the catalyst composition, provide access of feed molecules to and removal of products from the molecular sieve, and have little to no effect on conversion and selectivity of the molecular sieve. It would therefore be desirable to have an improved molecular sieve catalyst composition having a better conversion rate, improved olefin selectivity and a longer lifetime.

Commercial practice of the processes typically involves the use of fixed bed reactors. The catalyst in the fixed bed reactor deactivates as coke builds up in the catalyst. Without going into the details on reaction mechanisms, it is known that the use or presence of methanol can increase the rate of coke formation. Deactivated catalysts require oxidative regeneration to burn off the accumulated coke. Regeneration can be achieved by removing the catalyst from the reactor and burning off the coke in a regenerator unit or by isolating the reactor from methanol feed and introducing air to burn off coke under appropriately controlled conditions. After regeneration, the regenerated catalyst is reintroduced into the methanol conversion process or, more typically, the bed is put back on-line for methanol conversion. It is therefore desirable to have longer catalyst lifetime. The longer the catalyst lifetime, the less frequently the regeneration takes place, which leads to less investment for the process.

### SUMMARY OF THE INVENTION

We have now found that catalyst lifetime on cycle may be significantly increased by forming the catalyst with a mixture of a zeolitic methanol conversion catalyst with a basic metal oxide co-catalyst. The improvement in catalyst cycle life is achieved, moreover, without significantly affecting the reaction selectivity towards to desired hydrocarbon product(s).

The methanol conversion process according to the present invention comprises contacting a feedstream comprising methanol, optionally with dimethyl ether or other oxygenates with a catalyst comprising a physical mixture of a molecular sieve which may be a zeolite such as an MFI zeolite, with a basic metal oxide.

Suitable basic metal oxide co-catalysts which may be used for this purpose include magnesium oxide, calcium oxide and other alkaline earth metal oxides as well as oxides of the rare earth elements including cerium, the metals of the lanthanide series and the chemically similar elements scandium and yttrium, of which yttrium is preferred. The metal oxides may be supported on a porous support, e.g. a porous support of another metal oxide. This option is favored so as to improve the dispersion of the active metal oxide(s) making a greater number of active sites available for intercepting the formaldehyde which acts as a precursor in the formation of the coke which eventually deactivates the catalyst.

The preferred molecular sieve materials are zeolites are the small or medium pore size zeolites, preferably the medium pore (10-membered ring) zeolites exemplified by the zeolites of MFI structure such as ZSM-5 and ZSM-11; small pore (8-membered ring) zeolites such as chabazite, erionite, zeolite 4A, but non-zeolitic molecular sieves such as the silicoaluminophosphates (SAPOs) and aluminophosphates (ALPOs) may also be used.

### DRAWINGS

The single Figure of the accompanying drawings is a graph showing the conversions profiles for the methanol to gasoline (MTG) reaction for ZSM-5 and ZSM-5/yttria, as described below.

### DETAILED DESCRIPTION

The conversion of methanol or methanol/DME mixtures to olefins, gasoline and other hydrocarbons is effected by contacting the methanol-containing feed with the olefin-forming catalyst to form the desired hydrocarbon product, particularly ethylene and propylene but may also be higher olefins such as butane, hexane or octane or gasoline or distillate boiling range hydrocarbons. The process for converting the oxygenate feedstock is preferably a continuous fluidized-bed process to minimize the problems associated with the reaction exotherm although fixed bed operation, preferably using recycle or feed diluent to carry off reaction heat is not excluded.

The present catalyst system is useful for the various reactions in which methanol of mixtures of methanol with dimethyl ether or other oxygenates are converted to hydrocarbons. Generally these reaction schemes have been classified as methanol-to-gasoline (MTG), methanol-to-olefins (MTO), methanol-to-chemicals (MTC) (which actually is methanol-to-olefins since olefins are the predominant and desired product), methanol-to-aromatics (MTA) and combination processes such as the MTO/OCP Process mentioned above as well as for the production of gasoline and distillate by a combination of the methanol-to-olefins (MTO) and Mobil Olefins to Gasoline and Distillate Process (MOGD) or even to lubes production by a combination of MTO with the Mobil Olefins to Gasoline, Distillate and Lubes Process (MOGDL) as noted in U.S. Pat. No. 4,678,645. By selection of suitable empirically determined operating parameters, the products can be varied according to the needs of the operator.

The reaction processes can take place in a variety of catalytic reactors such as hybrid reactors that have dense bed or fixed-bed reaction zones and/or fast fluidized-bed reaction zones coupled together, circulating fluidized-bed reactors, riser reactors, and the like. Suitable conventional reactor types are described in, for example, U.S. Pat. Nos. 4,076,796 and 6,287,522 (dual riser), and Fluidization Engineering, D. Kunii and O. Levenspiel, Robert E. Krieger Publishing Company, New York, N.Y. 1977.

One preferred reactor type is a riser reactor. These types of reactors are generally described in Riser Reactor, Fluidization and Fluid-Particle Systems, pp. 48 to 59, F. A. Zenz and D. F. Othmo, Reinhold Publishing Corporation, N.Y., 1960, and U.S. Pat. No. 6,166,282 (fast-fluidized-bed reactor).

The average reaction temperature employed in the conversion process, specifically within the reactor, is typically from about 250° C. to about 600° C. Preferably the average reaction temperature within the reactor is from about 250° C. to about 500° C.; more preferably, from about 300° C. to about 500° C. The pressure employed in the conversion process, specifically within the reactor, is not critical. The reaction pressure is based on the partial pressure of the feedstock exclusive of any diluent therein. Typically, the reaction pressure employed in the process is in the range of from about 0.1 kPaa to about 5 MPaa, preferably from about 5 kPaa to about 1 MPaa, and most preferably from about 20 kPaa to about 500 kPaa.

In the fluidized bed process, the weight hourly space velocity (WHSV), defined as the total weight of the feed excluding any diluents to the reaction zone per hour per weight of molecular sieve in the molecular sieve catalyst composition in the reaction zone, is maintained at a level sufficient to keep the catalyst composition in a fluidized state within a reactor. Typically, the WHSV ranges from about 1 hr-1 to about 5000 hr-1, preferably from about 2 hr-1 to about 3000 hr-1, more preferably from about 5 hr-1 to about 1500 hr-1, and most preferably from about 10 hr-1 to about 1000 hr-1. In one preferred embodiment, the WHSV is greater than 20 hr-1, preferably the WHSV for conversion of a feedstock containing methanol and dimethyl ether is in the range of from about 20 hr-1 to about 300 hr-1. The superficial gas velocity (SGV) of the feedstock, including diluent and reaction products within the reactor, is preferably sufficient to fluidize the molecular sieve catalyst composition within a reaction zone of the reactor. The SGV in the process, particularly within the reactor system, more particularly within a riser reactor, is at least 0.1 meter per second (m/sec), preferably greater than 0.5 m/sec, more preferably greater than 1 m/sec, even more preferably greater than 2 m/sec, yet even more preferably greater than 3 m/sec, and most preferably greater than 4 m/sec. The specific reaction parameters to be used in the process can be selected by the skilled engineer according to the teachings in this art and experience with these reactions and the process units being used.

Product and other gases are withdrawn from the reactor and are passed through a recovery system. Any conventional recovery system, technique and/or sequence useful in separating olefin(s) and purifying olefin(s) from other gaseous components can be used in this invention. Examples of recovery systems include one or more or a combination of various separation, fractionation and/or distillation towers, columns, and splitters, and other associated equipment; for example, various condensers, heat exchangers, refrigeration systems or chill trains, compressors, knock-out drums or pots, pumps, and the like.

The catalyst used for the methanol conversion reaction is a physical mixture of the selected molecular sieve, preferably a zeolite and a basic metal oxide. As noted above, the preferred zeolites are the intermediate pore size zeolites exemplified by the zeolites of MFI structure such as ZSM-5 and ZSM-11 and small pore size zeolites such as erionite, zeolite 4A, and zeolites of the CHA and ITE structural types; zeolites of other structures have not been demonstrated to be as effective and accordingly are not preferred. Non-zeolitic molecular sieves such as the SAPOs and ALPOs may also be used, preferably the small pore sieves such as SAPO-18 and SAPO-34. Zeolites should have a silica:alumina ratio of at least 10:1 and preferably of at least 50:1, 100:1 or higher in order to enable it to resist the deactivating effect of the high temperature steam which is released during the methanol dehydration reaction. Ratios of 200:1, 500:1 or even higher (although with some structural aluminum to confer the desired activity) may be used. The zeolite should be at least partially in the H-form. Zeolite crystal size ranges from less than 0.05 micron to 5 micron, with a preferred range between 0.5 and 2 micron; the crystals can be present in larger agglomerates.

Non-zeolitic molecular sieves may also be found to be effective as catalysts. Non-zeolitic molecular sieve include the silicoaluminophosphate (SAPO) and aluminophosphate (ALPO), materials and mixtures of them, preferably, the SAPOs. Small pore non-zeolitic molecular sieves are defined as having a pore size of less than about 0.5 nm. Generally, suitable catalysts have a pore size ranging from about 0.35 to about 0.5 nm, preferably from about 0.40 to about 0.50 nm, and most preferably from about 0.43 to about 0.50 nm.

Non-zeolitic materials have been demonstrated to have catalytic properties for various types of conversion processes. Non-zeolitic molecular sieves are complex three dimensional crystalline structures which include either ALO₂ or SiO₂ or both AlO₂ and SiO₂ and a third metal oxide. The interstitial spaces or channels formed by the crystalline network enable non-zeolites to be used as molecular sieves as catalysts for chemical reactions and catalyst carriers in a wide variety of conversion processes with hydrocarbon feeds or products.

SAPO's have a three-dimensional microporous crystal framework structure of PO₂⁺ AlO₂⁻ and SiO₂ tetrahedral units. The chemical composition (anhydrous) is:

mR:(SiₓAL_{y}P_{z})O₂

where “R” represents at least one organic templating agent present in the intracrystalline pore system: “m” represents the moles of “R” present per mole of (SiₓAL_{y}P_{z})O₂ and has a value of from zero to 0.3, the maximum value in each case depending upon the molecular dimensions of the templating agent and the available void volume of the pore system of the particular SAPO species involved, and “x”, “y”, and “z” represent the mole fractions of silicon, aluminum and phosphorus, respectively. Typical small pore SAPO's are SAPO-17, SAPO-18, SAPO-34, SAPO-44, SAPO-56, and others. “R” may be removed at elevated temperatures.

ALPO's have a three-dimensional microporous crystal framework structure of PO.sub.2.sup.+ and AlO.sub.2.sup.− tetrahedral units. The chemical composition (anhydrous) is:

mR:(Al_{y}P_{z})O₂

where “R” represents at least one organic templating agent present in the intracrystalline pore system: “m” represents the moles of “R” present per mole of (Al_{y}P_{z})O₂ and has a value of from zero to 0.3, the maximum value in each case depending upon the molecular dimensions of the templating agent and the available void volume of the pore system of the particular SAPO species involved, and “y” and “z” represent the mole fractions of aluminum and phosphorus, respectively. “R” may be removed at elevated temperatures.

The process of making the catalyst in-situ may be accomplished through any one of the standard synthetic methods including, but not limited to, hydrothermal synthesis under autogenic pressure at elevated temperatures. Typical precursors include, but are not limited to, aluminum oxide, aluminum trimethoxide, and aluminum triethoxide as the source of aluminum. Orthophosphoric acid, trimethyl phosphate, and triethyl phosphate are examples of typically used precursors for phosphorus. Colloidal silica, silica sol, silicon tetramethoxide, and silicon tetraethoxide are examples of typically used precursors for silica. Templates which are often used in the synthesis process, include, for example, tetramethylammonium hydroxide and tetraethylammonium hydroxide. The resultant catalyst mixture is stirred as required. In some cases, stirring is not required and the mixture may be left undisturbed for a time adequate to permit the desired level of incorporation. The catalyst product is finally filtered, optionally washed, dried, and calcined by conventional methods.

The basic metal oxide functions as a co-catalyst for the zeolite by affecting the chemistry for methanol conversion. It intervenes mainly in the coke formation by intercepting coke precursors such as formaldehyde so as to reduce coke selectivity, slowing it down or mitigating coke formation and with reduced coke formation, inherently creating a more active methanol conversion catalyst.

The metal oxide co-catalyst used in conjunction with the zeolite is a metal oxide having basic characteristics, of which the oxides of the alkaline earth metals, such as calcium oxide and magnesium oxide. Basic oxides of the metals of the oxides of the rare earth elements including cerium, the metals of the lanthanide series and the chemically similar elements scandium and yttrium are preferred and of these, yttrium oxide is preferred. The basic metal oxide may itself be supported on a porous inorganic support material such as a porous inorganic oxide or mixture of oxides, preferably one which is basic or neutral in character so as not to impose any undesired competing reactions. The function of the support is to improve the dispersion of the active metal oxide(s) so a greater number of active sites are available for intercepting formaldehyde and to this end, high dispersion and high surface area are desirable attributes . Suitable porous metal oxide supports include zirconia (ZrO₂), titania (TiO₂), silica (SiO₂), ceria (CeO₂), magnesia (MgO), monohydrocalcite or non-acidic aluminas. The amount of support relative to the active basic metal oxide should be about 50 weight percent with lower amounts being preferred, e.g. 5, 10, 20 or 25 wt. pct. being highly suitable in order to optimize the amount of the active metal oxide; in each case, the amount of support will be selected according to the surface area and porosity of the support and its ability to disperse the active oxide in proximity to the sieve. A specific example of a supported active metal oxide is 5-10 wt.pct. La₂O₃/ZrO₂.

It is important to use a physical mixture of the basic metal oxide and the zeolite for the present purposes as opposed to incorporation of the metal into the zeolite structure, or i.e. the internal pore structure of the zeolite. For this reason, addition of the metal oxide component by ion-exchange with the zeolite or by wet impregnation onto an extruded catalyst which also results in exchange are not suitable for the present purposes. The physical mixture may comprise a blended but unconsolidated mixture or, more conveniently, an extrudate of the oxide and the zeolite possibly with a binder such as a clay to maintain coherence of the extruded particles. A separate particle catalyst system is envisaged with the metal oxide and the zeolite in separate particles, particularly in moving bed or fluidized bed operation in which extruded catalysts combined with a binder are favored for attrition resistance. When using a binder, the zeolite component and the basic metal oxide component may be in separate particles or combined into a single particle catalyst. Binders should be selected to be non-acidic and if separate particle catalyst systems Particles in moving bed operation may be in the 0.5-2 cm size range and particles for fluid bed operation in the conventional size range for this technology, typically from 10 to 100 microns with 50 to 100 microns being preferred.

The weight ratio of the zeolite to the metal oxide is typically from 50:50 to 90:10 although variations outside this range may be permissible depending upon the reaction conditions selected.

### EXAMPLES 1-2

The invention is illustrated using the MTG process. The MTG catalyst used in this investigation was a Zeolyst™ HZSM-5 with Si/Al ratio of 280). Example 1 was a control experiment using HZSM-5 as the catalyst; Example 2 used a catalyst composition of HZSM-5 and Y₂O₃ (80:20 w/w).

For Example 2, the HZSM-5 was intimately mixed with yttrium oxide powder using a mortar and pestle to form a catalyst composition with a percentage composition (HZSM-5:yttrium oxide) in the finished catalyst composition of 80: 20, by weight.

MTG Experiments were performed with the use of a quartz microflow TEOM reactor (tapered element oscillating microbalance reactor). Typically, ca. 10 mg of the catalyst was mixed with 25 mg of 100 micron quartz sand. The catalyst was loaded into the reactor. The reactor temperature was increased to 400° C. while the catalyst was under He flow (45 ml/min), a wait of ca. 40 min was allowed for the temperature to stabilize. Methanol was introduced into the catalyst at 84 microlitre/min at 400 WHSV and 170 kPag (25 psig) while the effluent was sampled by a 16-loop Valco™ valves. Typically, samples were analyzed to obtain the weighed average selectivity. The collected effluent samples were analyzed by an on-line gas chromatograph (Hewlett Packard 6890) equipped with a flame ionization detector. The chromatographic column used was a Q-column.

The weighted average yields from the tow runs were calculated based on the following formula:

x1*y1+(x2−x1)*y2+(x3−x2)*(y2+y3)/2+(x4−X3)*(y3+Y4)/2+ . . . ,

where xi and yi are yield and g methanol fed/g sieve, respectively.

Selectivities were calculated by normalizing the yield data excluding methanol and DME.

Quantification of the extension in catalyst life was determined by the Lifetime Enhancement Index (LEI) as defined by the following equation:

$$LEI = \frac{Lifetime of Catalyst in Combination with Metal Oxide}{Lifetime of Catalyst}$$

where the lifetime of the catalyst or catalyst composition, in the same process under the same conditions, is the cumulative amount of feedstock processed per gram of catalyst composition until the conversion of feedstock by the catalyst composition falls below some defined level, for example 1%.

The results of Examples 1 and 2 are summarized in Table 1 below in which the designations C1, C2═, C2°, C3═, C3°, C4s, C5-7 and aromatics refer to methane, ethylene, ethane, propene, propane, butenes and butanes, non-aromatic hydrocarbons that contain five to seven carbons, and aromatics, respectively. “Others” is the sum of H₂, CO and coke selectivity.

TABLE 1
Summary of lifetime enhancement index for HZSM-5 and HZSM-5/Y₂0₃

| | | | | | | | C5- | | | |
|---|---|---|---|---|---|---|---|---|---|---|
| | CH4 | C2= | C2 ° | C3= | C3 ° | C4s | 7s | Aromatics | Others | CMCPS |
| | Wt % | Wt % | Wt % | Wt % | Wt % | Wt % | Wt % | Wt % | Wt % | (1) |
| HZSM-5 | 0.8 | 6.7 | 0.1 | 29.6 | 1.1 | 20.4 | 35.1 | 5.6 | 0.6 | 312.0 |
| HZSM- | 0.7 | 5.7 | 0.0 | 29.7 | 0.9 | 20.2 | 36.8 | 4.2 | 1.9 | 462.3 |
| 5/Y203 | | | | | | | | | | |

(1) CMCPS Cumulative methanol converted per gram of sieve (g methanol/g catalyst HZSM-5), is a measure of catalyst lifetime in a single cycle.

The lifetime of the HZSM-5 catalyst (Example 1) was measured to be 312 g methanol converted/g sieve.

The lifetime of the HZSM-5/Y203 catalyst composition (Example 2) was measured to be 462.3 g methanol converted/g sieve.

The LEI for the catalyst composition is 1.5. In other words, as a result of the introduction of yttrium oxide, there is a 50% increase of catalyst lifetime in the methanol conversion process.

The Figure compares the conversion profiles for HZSM-5 alone and HZSM-5/Y₂O₃. Note that the catalyst composition containing Y₂O₃ typically has comparable or higher activity. This is particularly true towards the end of the runs.

1. A methanol conversion process which comprises contacting a feedstream comprising methanol under methanol conversion conditions with a catalyst comprising a physical mixture of molecular sieve with a basic metal oxide to form a hydrocarbon product.

2. A process according to claim 1 in which the molecular sieve comprises an intermediate pore size zeolite selected from an MFI or MEL zeolite.

3. A process according to claim 2 in which the intermediate pore size zeolite is ZSM-5 or ZSM-11

4. A process according to claim 1 in which the molecular sieve comprises a small pore size zeolite.

5. A process according to claim 1 in which the molecular sieve comprises chabazite.

6. A process according to claim 1 in which the basic metal oxide is an oxide of a metal of the lanthanide series.

7. A process according to claim 1 in which the basic metal oxide is an oxide of magnesium, calcium, cerium or scandium.

8. A process according to claim 1 in which the basic metal oxide is yttrium oxide.

9. A process according to claim 1 in which the molecular sieve comprises an intermediate pore zeolite selected from HZSM-5 or HZSM-11 and the basic metal oxide is yttrium oxide.

10. A process according to claim 1 in which the molecular sieve comprises an intermediate pore zeolite in a weight ratio of the intermediate pore size zeolite to the basic metal oxide from 50:50 to 90:10.

11. A process according to claim 10 in which the weight ratio of the intermediate pore size zeolite to the basic metal oxide is from 60:40 to 80:20.

12. A process according to claim 1 in which the catalyst is a composite particle catalyst comprising the molecular sieve and the basic metal oxide together in the same particles.

13. A process according to claim 12 in which the composite particle catalyst comprises extrudates of the molecular sieve and the basic metal oxide together in the same extrudate particles.

14. A process according to claim 1 in which the catalyst is a separate particle catalyst system comprising the molecular sieve and the basic metal oxide in separate particles.

15. A process according to claim 14 in which the separate particle catalyst comprises extrudates of the molecular sieve and extrudates of the basic metal oxide as separate extrudate particles.

16. A process according to claim 1 in which the catalyst is maintained in a fixed bed.

17. A process according to claim 1 which is operated as a moving bed process.

18. A process according to claim 1 which is operated as a fluidized bed process.

19. A process according to claim 1 in which the feedstream comprises methanol and another oxygenate.

20. A process according to claim 1 in which the feedstream comprises methanol and dimethyl ether.

21. A process according to claim 1 in which the hydrocarbon product comprises olefins or aromatics.
